(19) United States
(12) Patent Application Publication
(10) Pub. No.: US 2007/0036840 A1
(43) Pub. Date: Feb. 15, 2007

(54) Phosphorylated glucomannan polysaccharides containing 1-6 and 1-2 linkages increase weight gain in swine

(76) Inventors: Jose Antonio Matji Tuduri, Madrid (ES); Antonio F. Guerrero Gomez-Pamo, Madrid (ES); Jose Luis Alonso Lebrero, Madrid (ES); Garrett Lindemann, Sheridan, WY (US)

Correspondence Address: LATHROP & GAGE LC, 4845 PEARL EAST CIRCLE, SUITE 300, BOULDER, CO, 80301, US

(21) Appl. No.: 11/490,716

(22) Filed: Jul. 20, 2006

Related U.S. Application Data
(60) Provisional application No. 60/702,885, filed on Jul. 27, 2005.
(60) Provisional application No. 60/703,028, filed on Jul. 27, 2005.
(60) Provisional application No. 60/702,886, filed on Jul. 27, 2005.
(60) Provisional application No. 60/702,878, filed on Jul. 27, 2005.
(60) Provisional application No. 60/702,887, filed on Jul. 27, 2005.

(51) Int. Cl. A61K 31/715; A23K 1/165

(52) U.S. Cl. 424/442; 514/54

(57) ABSTRACT

Phosphorylated glucomannans may be purified from naturally occurring sources and used as a supplement to swine feeds for the benefit of swine production.

### RELATED APPLICATIONS

This application claims benefit of priority to provisional application Ser. No. 60/702,885 filed Jul. 27, 2005, provisional application Ser. No. 60/703,028 filed Jul. 27, 2005, provisional application Ser. No. 60/702,886 filed Jul. 27, 2005, provisional application Ser. No. 60/702,878 filed Jul. 27, 2005, and provisional application Ser. No. 60/702,887 filed Jul. 27, 2005.

### BACKGROUND

1. Field of the Invention

This disclosure pertains to the supplementation of swine diet with phosphorylated glucomannan polysaccharides to the benefit of swine production. Benefits may include, for example, an increased rate of weight gain and decreased mortality in a population of swine.

2. Description of the Related Art

Antibiotics may be added to the nursery, grower and finisher feeds of swine to promote growth and/or reduce disease occurrence during all phases of food production. The purpose for addition of the antibiotics is to promote growth during the starter, grower and finishing phase of monogastric animal production¹. The antibiotics promote growth through the reduction of biological stress, the decrease of malicious bacteria, and by promoting the health of the swine. Swine that are healthy and disease free eat more food, and more effectively convert the food into muscle or meat. Typically, subtherapeutic levels of antibiotics increase growth rate about 15% and improve efficiency of feed conversion 5 to 7%. On the other hand, swine that are unhealthy or not disease free, are stressed. Relatively more of the ingested fed energy is utilized to reduce or remove the biological stress the animal is facing. Thus, the antibiotic supplementation of swine diet is shown to have numerous benefits.

Despite these advantages, the practice of supplementing swine diet with antibiotics is increasingly problematic. Sub-therapeutic doses of antibiotics are linked to the increased presence of antibiotic-resistant bacterial strains in humans, animals and in the environment^{2,3}. It is also possible for residual antibiotics to appear in food that is meant for human consumption. The United States Food and Drug Administration (USFDA) requires the antibiotic must be with drawn from the feed of the swine at least two weeks prior to slaughter to prevent the antibiotics sequestered in the swine from being ingested by humans.

The problems resulting from subtherapeutic antibiotic usage are of such growing significance that various other regulatory agencies have taken keen interest. In one example of a regulatory response, the European Union has recently mandated that antibiotics may not be used as growth promoters in feed animals⁴. Over the years, antibiotics have been slowly restricted, culminating with the complete banning of antibiotics in the European Union as growth promoters commencing Jan. 1, 2006.

The restriction or banning of antibiotic supplements to animal diets has direct cost in terms of economics and animal health. The commercial cost of producing meat and milk from animals has increased and the health of the animals in high density production facilities has decreased^{1,2}.

One alternative to the use of antibiotics as growth promoters includes oligosaccharide products that are derived from yeast cell walls and are composed of sugars such as galactose, fructose, and mannose¹. These small fragments of carbohydrates may selectively stimulate some of the gut flora of an animal. This stimulation alters the microbial balance, resulting in a benefit to the host animal³. Additionally, the animal may not digest some of the small fragments of carbohydrates. As one example, mannan oligosaccharides are not digested by poultry, and pass through the animal functioning as a soluble fiber. One benefit of this type of soluble fiber is a cleansing effect by detaching pathogens from the animal's gut^{5,1,3}, thereby removing the pathogens from the animal's gastrointestinal tract.

Growth promotion in broiler, chickens and turkeys by mannan oligosaccharide has been investigated and demonstrated to be effective. Studies indicate that inclusion of a commercially available mannan oligosaccharide, Bio-Mos®, in broiler diets allows the broilers to perform similar to broilers fed the same diet containing antibiotics on the parameters of feed conversion, weight gain, parts yield, dressing percentage and mortality⁶. Turkeys fed a diet containing Bio-Mos® (0.10%) performed as well as did turkeys fed a control diet containing an antibiotic. Parameters measured for comparison between groups included; intestinal breaking strength, body weight, mortality, breast meat yield, and feed conversion⁷.

Another study concluded that turkeys fed a diet containing a concentration of mannan oligosaccharides out performed the control groups and led to the conclusion that mannan oligosaccharides may be used as an alternative to antibiotics as a growth promotant to improve turkey performance⁸. Weanling swine diets containing mannan oligosaccharides or phosphorylated mannan oligosaccharides have been demonstrated to have a growth promoting effect^{9,10,11}. Additional research has indicated that supplementing a dry cow's diet with mannan oligosaccharide enhances the cow's response to rotavirus and tends to enhance the transfer of those rotavirus antibodies to claves¹². Furthermore, feeding fructooligosaccharide, mannanoligosaccharide, oligofructose and Inulin have been demonstrated to protect mice¹³ from enteric and systemic pathogens and tumor inducers as well as increase the immune status and colonic health of dogs¹⁴.

One benefit of feeding mannan oligosaccharides to chickens is the growth promotion of bacteria that are beneficial to the host; namely and as an example, species of Bifidobacterium and Lactobacillus; while decreasing the colonization and growth of unbeneficial bacterial species to the host; namely and as an example species of Enterbacteriaceae, Enterococcus and Salmonella^{15,16}.

In general, oligosaccharides, specifically the mannan family of carbohydrates, have been demonstrated to be potent immunostimulants; activating macrophages, stimulating T-cells and blocking phagocytosis. The response is elicited through the binding of the mannan to receptors that are located on the macrophage external surface and intercellularly^{17,18}. Acemannan (ACM 1) is a β-(1-4)-acetylated mannan isolated from Aloe vera that has been used in wound healing and as an adjuvant in vaccinations¹⁹. Delivery of a single low dose of ACM 1 to a chicken by intramuscular injection has been demonstrated to result in a systemic immuno-modulated activation of macrophages¹⁹.

One example of an immune enhancing glucomannan reported in U.S. Pat. No. 4,138,479 issued to Truscheit, et al., which teaches the use of a glucomannan protein that is purified from yeast cells. An extraction protocol contacts the yeast with equal parts of phenol and water. Three phases including solids, phenol and water are separated by centrifugation. The aqueous phase is concentrated by dialysis and then lyophilized. The resulting solid composition induces an immunopotentiating response and so are somewhat effective against neoplasms.

Other glucomannans from aloe have been reported to have an immunopotentiating function. U.S. Pat. No. 6,271,214 issued to Qiu et al. describes the concentration of β-1,4 glucomannan from aloe by a combination of hydrolysis and chromatography. The β-1,4 glucomannan is useful as an immunomodulating or immunostimulating composition, and may be administered topically or orally to treat radiation and chemically induced swelling of murine ear tissues.

A phosphorylated glucomannan, in combination with a seed coat protein that is commonly known as Immunoferon or AM3 has been demonstrated to stimulate haemolytic plaque-forming B lymphocytes²⁰ as well as enhancing the number and activity of peripheral blood monocytes and macrophages, and cytotoxic activities of NK cells in humans exhibiting indications of chronic bronchitis and mice of an elderly age²¹. Further, the ability of Immunoferon to restore natural killer (NK) cell phagocytic cells to normal activity has been verified in humans²².

Additionally, Immunoferon, not only activities and restores not only monocyte and macrophage cell function, but it also functions to reduce inflammation and inflammatory pathway activators. Specifically, Immunoferon has been demonstrated to reduce proinflammatory molecules such as Tumour Necrosis Factor α (TNF-α)²³. In the case of lipopolysaccahride induced TNF-α, research demonstrated that treatment with Immunoferon resulted in regulation of TNF-α through increased production of TNF-α such as Interleukin 10 (IL-10) and corticosteriods as well as the inhibition of Interleukins 1 and 6 (IL-1 and IL-6)²⁴. Expression of these three cytokines, TNF-α, IL-6 and IL-1, alters the metabolism of the swine resulting in less than optimal weight gain, development and health²⁵.

Not all mannans have immunostimulatory activity. The mannans including disaccharide through hexasaccharide, released by weak alkaline degradation of the cetyltrimethulammonium bromide (CTAB) extraction of Candida albicans, do not demonstrate any immunostimulatory activity. In fact, these small mannans are potent inhibitors of lymphoproliferation²⁶.

Although various research has investigated the supplementation of swine diets, it has been previously unknown to supplement swine diet with glucomannan compositions. Generally, swine diets U.S. Pat. No. 5,480,659 issued to Tokach et al. describes the beneficial effect of supplemental valine in a soy-based diet. U.S. Pat. No. 4,746,531 issued to Lush describes a transitional diet including animal protein products, marine products, milk products, grain products, plant protein products, processed grain by-products and natural and artificial flavors in proportions to balance a corn-based feed ration for protein, fiber, energy and palatability.

### SUMMARY

The present instrumentalities overcome the problems outlined above and advance the art by providing a glucomannan composition that may be added to swine diets for the benefit of swine production. In one example, the glucomannan composition may be used to replace the subtherapeutic doses of antibiotics that are currently used in production swine feeds. The glucomannan composition may be mixed with nursery, grower or finishing feeds.

Preferred forms of glucomannan for use in supplementing swine diet include phosphorylated glucomannan polysaccharides. Particularly preferred forms are characterized by a subunit that is repeated approximately 30 to 40 times. The subunit contains 1-6 and 1-2 linkages between and within mannose and glucose residues at a ratio of 12:1 mannose:glucose, where also the phosphorylated glucomannan polysaccharide predominantly exists in the homo-trimeric form of an alpha helix. These phosphorylated glucomannans may be isolated from Candida utilis according to a protocol disclosed below.

In other aspects, the phosphorylated glucomannan polysaccharaides may be administered to swine in two basic forms, namely, phosphorylated glucomannan or phosphorylated glucomannan that is non-covalently linked to a protein. The phosphorylated glucomannan, with or without a non-covalently linked protein, may be adsorbed into a matrix. Without limitation, specific examples of absorption matrices include one or more inorganic salts, such as dihydrate calcium phosphate (CaHPO₄.2H₂O) and dihydrate calcium sulphate (CaSO₄.2H₂O). Phosphorylated glucomannan, with or without the non-covalently linked protein, absorbed or unabsorbed into a matrix, may be administered to the swine, preferably, if the form of a dry powder thoroughly mixed into the nursery, grower or finishing feeds.

Benefits of administering the phosphorylated glucomannan compositions to animals, especially swine, may include:
Increased animal weight gain;
Increased relative quantities of the beneficial bacteria in the animal;
Decreased relative quantities of malicious bacteria in the animal;
Increased uptake of beneficial minerals, nutrients and vitamins;
Increased uptake of zinc and copper;
Improved overall general health of the animal;
Replacement of subtherapeutic doses of antibiotics in animal feed; and/or
Reduced or eliminated subtherapeutic doses of antibiotics in animal feed.

A swine diet may be supplemented by mixing a conventional swine feed with a phosphorylated glucomannan polysaccharide in an effective amount to benefit swine production, in order to provide a mixed swine feed.

The phosphorylated glucomannan contains a repeating polysaccharide subunit that is repeated approximately n times of 1-6 and 1-2 linkages between and within mannose and glucose residues at a ratio of 12:1 mannose:glucose, were n ranges from 10 to 40. The value n may range from 10 to 20, from 20 to 30, from 30 to 40, or from 20 to 40, with n preferably being about 30.

The swine feed may be provided as a liquid, gel or colloid, for example, in the nature of a vitamin or mineral supplement. In other forms of what is disclosed, the feed is prepared as solid food, preferably with a balance of nutrients that target swine needs at a particular stage of swine development.

### DETAILED DESCRIPTION

According to one embodiment, the phosphorylated glucomannan is provided as an additive to swine feed that may be used at all stages of swine development. The phosphorylated glucomannan may, for example, be added and mixed into the feed as a concentrated raw product, a concentrated raw product with a non-covalently attached protein, raw product absorbed into a matrix, and/or a concentrated raw product with a non-covalently attached protein absorbed into a matrix.

The phosphorylated glucomannan may be in the form of a dry powder that is capable of being added to or mixed with swine feed. Dosing is by ratio or concentration that may vary according to the stage of swine development to provide a benefit to the swine by promoting the health of the swine and replacing, reducing or eliminating the use of subtherapeutic doses of antibiotics in swine nursery, grower, finisher and maintenance feeds.

Exemplary embodiments of various formulations include:
i) A dry powder comprised of the phosphorylated glucomannan polysaccharides containing a subunit, repeated approximately 30 times, of 1-6 and 1-2 linkages between and within mannose and glucose residues at a ratio of 12:1 mannose:glucose mixed into swine feed at a concentration, ratio, or dose that provides the general benefits of good health and weight gain to the swine consuming the mixed feed.
ii) A dry powder comprised of phosphorylated glucomannan polysaccharides containing a subunit, repeated approximately 30 times, of 1-6 and 1-2 linkages between and within mannose and glucose residues at a ratio of 12:1 mannose:glucose and a non-covalently linked protein mixed into swine feed at a concentration, ratio, or dose that provides the general benefits of good health and weight gain to the swine consuming the mixed feed.
iii) A dry powder comprised of the phosphorylated glucomannan polysaccharides containing a subunit, repeated approximately 30 times, of 1-6 and 1-2 linkages between and within mannose and glucose residues at a ratio of 12:1 mannose:glucose and absorbed into a matrix and mixed into swine feed at a concentration, ratio, or dose that provides the general benefits of good health and weight gain to the swine consuming the mixed feed.
iv) A dry powder comprised of phosphorylated glucomannan polysaccharides containing a subunit, repeated approximately 30 times, of 1-6 and 1-2 linkages between and within mannose and glucose residues at a ratio of 12:1 mannose:glucose and a non-covalently linked protein and absorbed into a matrix and mixed into swine feed at a concentration, ratio, or dose that provides the general benefits of good health and weight gain to the swine consuming the mixed feed.

The University of Georgia College of Agricultural & Environmental Sciences Cooperative Extension Service, in Bulletin 854/Revised May, 1995, identifies the following tables that may be used to formulate swine diets. These tables may be used to formulate ideal swine feeds for various stages of swine growth. Local variances in the content of various feed sources may be accounted for by laboratory food analysis to confirm the general guidelines presented below.

TABLE 1
Daily Nutrient Allowances of Breeding Swine

| | Sows and Gilts | | |
|---|---|---|---|
| Nutrient | Gestation | Lactation¹ | Boars |
| Metabolizable energy, kcal | 5,700² | 17,570³ | 7,000 |
| Protein, g | 236 | 763 | 318 |
| Lysine, g | 9.1 | 40.0 | 13.6 |
| Methionine + cystine, g | 5.8 | 24 | 8.1 |
| Tryptophan, g | 1.6 | 7.8 | 2.7 |
| Threonine, g | 6.4 | 28 | 10.0 |
| Calcium, g | 16.3 | 40.9 | 20.4 |
| Phosphorus, g | 13.6 | 32.7 | 17.0 |
| Salt, g | 6.4 | 19.1 | 7.9 |
| Iron, mg | 144 | 432 | 180 |
| Copper, mg | 10.8 | 32.4 | 13.5 |
| Manganese, mg | 56 | 168 | 70 |
| Zinc, mg | 120 | 360 | 150 |
| Iodine, mg | .4 | 1.2 | 0.5 |
| Selenium, mg⁴ | — | — | — |
| Vitamin A, IU | 12,000 | 36,000 | 15,000 |
| Vitamin D, IU | 1,200 | 3,600 | 1,500 |
| Vitamin E, IU | 50 | 150 | 62.5 |
| Vitamin K, mg | 4 | 12 | 5 |
| Riboflavin, mg | 8 | 24 | 10 |
| Niacin, mg | 48 | 144 | 60 |
| Pantothenic acid, mg | 40 | 120 | 50 |
| Choline, mg | 1,600 | 4,800 | 2,000 |
| Vitamin B12, mcg | 40 | 120 | 50 |
| Biotin, mg | .24 | .72 | 0.30 |
| Folic acid, mg | 1.8 | 5.4 | 2.25 |
| Feed required, lb | 4 | 12 | 5 |

¹To determine the requirement as a percentage or amount/lb of diet, use the following formulas:
g/day to %: (daily requirement/454)/daily feed intake, lb/day × 100 = %
mg/day to mg/lb: daily requirement/daily feed intake, lb/day = mg/lb
IU/day to IU/lb: daily requirement/daily feed intake, lb/day = IU/lb
²Research has shown that increasing daily energy intake during the last 10-30 days of gestation may improve birth weights and pig survival. Daily energy intake should be increased 718-2700 kcal/day depending on pig weights and survival.
³Daily energy needs depend on level of production. This level of energy is adequate for a sow nursing 8 pigs. Daily energy intake should increase 1500 kcal for each pig over 8.
⁴The maximum level of selenium allowed by FDA is subject to change. Check current feed regulations.

TABLE 2
Nutrient Allowances of Starting, Growing and Finishing
Swine
Percentage or Amount per pound of Diet

| — | Liveweight class, lb | | | | |
|---|---|---|---|---|---|
| Nutrient | <15¹ | 15-20² | 20-40 | 40-110 | 110-mkt. |
| Metabolize energy, kcal | 1,500 | 1,500 | 1,500 | 1,400 | 1,400 |
| Protein, % | 24 | 20 | 18 | 15 | 13 |
| Lysine, % | 1.40 | 1.25 | 1.15 | .80 | .65 |
| Methionine + cystine, % | .77 | .69 | .63 | .45 | .37 |
| Tryptophan, % | .22 | .20 | .18 | .13 | .11 |
| Threonine, % | .90 | .80 | .74 | .53 | .44 |
| Calcium, % | .95 | .90 | .85 | .65 | .60 |
| Phosphorus, % | .80 | .75 | .70 | .55 | .50 |
| Salt, %³ | .35 | .35 | .35 | .35 | .35 |
| Iron, mg | 45 | 45 | 45 | 36 | 36 |
| Copper, mg⁴ | 3.4 | 3.4 | 3.4 | 2.7 | 2.7 |
| Manganese, mg | 14.0 | 14.0 | 14.0 | 14.0 | 14.0 |
| Zinc, mg | 45.0 | 45.0 | 45.0 | 36.0 | 36.0 |
| Iodine, mg | 0.1 | 0.1 | 0.1 | 0.1 | 0.1 |
| Selenium, mg⁴ | 0.045 | 0.045 | 0.045 | 0.045 | 0.045 |
| Vitamin A, IU | 3,000 | 3,000 | 2,500 | 1,500 | 1,500 |
| Vitamin D, IU | 300 | 300 | 250 | 150 | 150 |
| Vitamin E, IU | 10 | 10 | 10 | 8 | 8 |
| Vitamin K, mg | 1 | 1 | 1 | 1 | 1 |
| Riboflavin, mg | 2.5 | 2.25 | 2.0 | 1.4 | 1.2 |
| Niacin, mg | 12 | 12 | 10 | 8 | 8 |
| Pantothenic acid, mg | 8 | 8 | 8 | 7 | 6 |
| Choline, mg | 600 | 600 | 600 | 400 | 300 |
| Vitamin B12, mcg | 10 | 10 | 10 | 6 | 6 |

¹Pigs weaned at less than 28 days of age. This diet should contain 25-75% milk products.
²This diet should contain 5-25% milk products.
³When adding large amounts of dried whey (10% or more), the added salt should be reduced (.25%) due to the high salt content of some whey products.
⁴Addition of 57-114 mg/lb of copper from copper sulphate has been shown to improve performance of young pigs (up to 40 lbs).
⁵The maximum level of selenium allowed by FDA is subject to change. Check current feed regulations.

TABLE 3
Nutrient Allowances of Breeding Swine
Percentage or Amount per Pound of Diet

| | Sows and gilts | | |
|---|---|---|---|
| Nutrient | Gestation | Lactation¹ | Boars |
| Metabolize energy, kcal | 1,400 | 1,400 | 1,400 |
| Protein, % | 13 | 14 | 14 |
| Lysine, % | .50 | .80 | .60 |
| Menthionine + cystine, % | .32 | .44 | .36 |
| Tryptophan, % | .09 | .14 | .12 |
| Threonine, % | .35 | .51 | .44 |
| Calcium, % | .90 | .75 | .90 |
| Phosphorus, % | .75 | .60 | .75 |
| Salt, % | .35 | .35 | .35 |
| Iron, mg | 36 | 36 | 36 |
| Copper, mg | 2.7 | 2.7 | 2.7 |
| Manganese, mg | 14 | 14 | 14 |
| Zinc, mg | 30 | 30 | 30 |
| Iodine, mg | 0.1 | 0.1 | 0.1 |
| Selenium, mg² | 0.045 | 0.045 | 0.045 |
| Vitamin A, IU | 3000 | 3000 | 3000 |
| Vitamin D, IU | 300 | 300 | 300 |
| Vitamin E, IU | 12.5 | 12.5 | 12.5 |
| Vitamin K, mg | 1 | 1 | 1 |
| Riboflavin, mg | 2 | 2 | 2 |
| Niacin, mg | 12 | 12 | 12 |
| Pantothenic acid, mg | 10 | 10 | 10 |
| Choline, mg | 400 | 400 | 400 |
| Vitamin B12, mcg | 10 | 10 | 10 |
| Biotin, mg | 0.06 | 0.06 | 0.06 |
| Folic acid, mg | 0.45 | 0.45 | 0.45 |

¹The nutrient requirement of lactating sows increases as feed intake decreases. The nutrient content should be increased 8.3% for each pound below 12. (If sows will only consume 10 lb/day, the levels of required nutrients should be increased 16.6%.)
²The maximum level of selenium allowed by FDA is subject to change. Check current feed regulations.

TABLE 4
Average Analysis of Ingredients Used in Swine Diets¹

| | Metabolizable | | | | | | | Methionine | | |
|---|---|---|---|---|---|---|---|---|---|---|
| | energy | Protein | Calcium | Phosphorus | Fat | Fiber | Lysine | & cystine | Tryptophan | Threonine |
| Ingredient | kcal/lb | | | | percent | | | | | |
| Alfalfa meal | 600 | 17.5 | 1.40 | .26 | 3.2 | 23.5 | .80 | .58 | .36 | .70 |
| (dehydrated) | | | | | | | | | | |
| Animal fat | 3,590 | — | — | — | 99.4 | — | — | — | — | — |
| Barley | 1,275 | 11.5 | .06 | .36 | 1.8 | 7 | .36 | .37 | .16 | .42 |
| Blood meal | 1,200 | 80 | .28 | .22 | 1 | 1 | 5.37 | 2.44 | 1.02 | 4.90 |
| Calcium | — | — | 38.00 | — | — | — | — | — | — | — |
| carbonate | | | | | | | | | | |
| (limestone) | | | | | | | | | | |
| Corn | 1,500 | 8.2 | .01 | .24 | 3.6 | 2.2 | .24 | .39 | .09 | .39 |
| (yellow) | | | | | | | | | | |
| Cottonseed | 1,100 | 41.7 | .20 | 1.00 | 2.7 | 13.4 | 1.55 | 1.14 | .48 | 1.32 |
| meal | | | | | | | | | | |
| (Solvent) | | | | | | | | | | |
| Defluorinated | — | — | 32.00 | 18.00 | — | — | — | — | — | — |
| phosphate | | | | | | | | | | |
| Dicalcium | — | — | 20.00 | 18.50 | — | — | — | — | — | — |
| phosphate | | | | | | | | | | |
| Distillers | 1,270 | 27 | .35 | .95 | 8.0 | 8.5 | .60 | 1.00 | .20 | .92 |
| dried grains | | | | | | | | | | |
| & solubles, | | | | | | | | | | |
| corn | | | | | | | | | | |
| Fish meal | 1,200 | 61.5 | 5.10 | 2.98 | 9.7 | .7 | 4.60 | 2.50 | .71 | 2.50 |
| (menhaden) | | | | | | | | | | |
| Grain | 1,425 | 9.8 | .02 | .27 | 3.0 | 2.8 | .22 | .31 | .09 | .27 |
| sorghum | | | | | | | | | | |
| (milo) | | | | | | | | | | |
| Meat & bone | 1,100 | 50.6 | 8.90 | 3.93 | 10.8 | 2.4 | 2.50 | 1.27 | .29 | 1.50 |
| meal | | | | | | | | | | |
| Molasses, | 1,060 | 3 | .50 | .05 | — | — | — | — | — | — |
| cane | | | | | | | | | | |
| Oats | 1,200 | 12 | .10 | .33 | 4.0 | 12.0 | .40 | .33 | .13 | .16 |
| Oat groats | 1,450 | 15.8 | .08 | .43 | 6.2 | 2.5 | .45 | .46 | .18 | .20 |
| Peanut meal | 1,375 | 52.3 | .15 | .55 | 4.1 | 6.0 | 1.78 | 1.26 | .55 | .49 |
| Rye | 1,230 | 12.6 | .08 | .30 | 1.8 | 2.8 | .40 | .36 | .14 | .86 |
| Skim milk, | 1,545 | 33 | 1.25 | 1.00 | .5 | — | 2.70 | 1.20 | .45 | 1.60 |
| dried | | | | | | | | | | |
| Soybean | 1,520 | 49.2 | .40 | .65 | 1.5 | 3.3 | 3.20 | 1.55 | .63 | 1.92 |
| meal | | | | | | | | | | |
| (solvent, | | | | | | | | | | |
| hulled) | | | | | | | | | | |
| Soybean | 1,475 | 45.6 | .50 | .71 | 2.1 | 5.9 | 2.88 | 1.22 | .55 | 1.81 |
| meal | | | | | | | | | | |
| (solvent) | | | | | | | | | | |
| Soybeans | 1,600 | 38 | .25 | .58 | 18.0 | 5.0 | 2.40 | 1.05 | .55 | 1.50 |
| (whole | | | | | | | | | | |
| cooked) | | | | | | | | | | |
| Sunflower | 1,044 | 28.7 | .38 | .97 | 2.7 | 24.8 | 1.47 | 2.00 | .52 | 2.13 |
| meal | | | | | | | | | | |
| (extracted | | | | | | | | | | |
| w/hulls) | | | | | | | | | | |
| Meat meal | 1,020 | 59.5 | 5.80 | 2.99 | 9.0 | 2.2 | 3.73 | 1.19 | .72 | 2.39 |
| tankage | | | | | | | | | | |
| Triticale | 1,475 | 11.8 | .05 | .30 | 1.8 | 2.9 | .43 | .60 | .13 | .41 |
| Wheat (soft | 1,500 | 11.9 | .01 | .22 | 1.7 | 2.8 | .35 | .34 | .12 | .32 |
| winter) | | | | | | | | | | |
| Wheat bran | 890 | 15.0 | .14 | 1.10 | 4.1 | 11.0 | .50 | .27 | .27 | .42 |
| Wheat | 910 | 17.1 | .10 | .88 | 4.4 | 7.9 | .60 | .31 | .20 | .49 |
| middlings | | | | | | | | | | |
| Whey (dried) | 1,445 | 12 | .90 | .80 | .5 | — | .80 | .40 | .13 | .89 |
| Whey (low | 1,250 | 17.0 | 1.50 | 1.20 | 1.0 | — | 1.47 | 1.14 | .36 | .50 |
| lactose dried) | | | | | | | | | | |

¹Standard reference values modified to reflect Georgia feedstuffs.

TABLE 5
Maximum Levels of Ingredients for Swine Diets
Maximum level % in complete feed

| | | Grower- | | | |
|---|---|---|---|---|---|
| Ingredient | Starter | Finisher | Gestation | Lactation | Remarks |
| Alfalfa meal- | 0 | 5 | 50 | 10 | A |
| dehydrated | | | | | |
| Animal fat | 5 | 8 | 8 | 8 | B |
| Barley | 0 | 80 | 80 | 50 | C |
| Blood Meal | 5 | 5 | 5 | 5 | D |
| Cottonseed | 0 | 5-10 | 5 | 5 | E |
| meal | | | | | |
| Meat and bone | 0 | 5 | 10 | 10 | D |
| meal | | | | | |
| Oats | 10 | 20 | 70 | 15 | C |
| Peanut meal | 0 | 5 | 5 | 5 | F |
| Rye | 0 | 20 | 20 | 20 | G |
| Tankage | 0 | 5 | 5 | 5 | D |
| Wheat bran | 0 | 0 | 30 | 10 | C |
| Wheat | 5 | 10 | 30 | 10 | C |
| middlings | | | | | |

These feeds may be supplemented with minor amounts of a phosphorylated glucomannan, for example, as isolated from Candida utilis, to achieve the instrumentalities described herein. Other feed formulations may be provided by publicly available software, such as the User-Friendly Feed Formulation Program (“UFFDA”) based upon the book Animal Feed Formulation—Economics and Computer Applications, by G. M. Pesti and B. R. Miller, Chapman and Hall. The phosphorylated glucomannan mixed with this food to provide a dosage ranging from 1 to 5 mg of the phosphorylated glucomannan per kg of body weight in the swine. The preferred dosage is 3 mg per kg of body weight Although higher doses may be used, such as doses of 20 mg/kg, the range from 1 mg to 5 mg per kg are generally minimal doses to achieve the desired effects.

### EXAMPLE 1

### Obtention of Candida Utlis Polysaccharide with Soy Protein Adsorbed on Calcium Phosphate

The following laboratory-scale example teaches by way of example how to purify a phosphorylated glucomannan polysaccharide. The polysaccharide is characterized by a subunit that is repeated approximately 30 to 40 times, where the subunit contains 1-6 and 1-2 linkages between and within mannose and glucose residues at a ratio of 12:1 mannose:glucose, where also the phosphorylated glucomannan polysaccharide predominantly exists in the homo-trimeric form of an alpha helix. The polysaccharide may be obtained, for example, using the process described in EP1163911, which is incorporated by reference, and describes the alternative use of soy or castor beans which are optionally omitted.

The method of isolating phosphorylated glucomannan polysaccharides commences, for example, by soaking soybeans in water to provide soaked soybeans. These are ground to provide ground material and combined with Candida utilis, water, and a first salt to provide an incubation mixture. The incubation mixture is incubated with stirring or agitation for extraction of the polysaccharide to provide a supernatant fluid. The supernatant is concentrated by filtration with a cutoff of about 20 kDa. A second salt is added together with a low molecular weight ketone to form a precipitate. The precipitate is dried to yield an isolated polysaccharide product.

In various aspects, the drying step is preferably performed at a temperature not more than 55° C. to avoid product degradation. The first salt is preferably a magnesium salt, such as MnSO₄.H₂O. The incubation mixture may be provided with an amount of camphor that is miscible with the aqueous phase, and with heating to a temperature of from 30° C. to 40° C. Concentration may be staged, for example, using an initial stage of filtering to remove cellular debris, ultrafiltration to the 20 kDA cutoff to produce a concentrate of at least 1/10 the initial volume of the supernatant, and diafiltration of the concentrate against water in amount at least ten times the volume of the concentrate. The second salt is preferably a calcium salt, such as calcium chloride, where also the low molecular weight ketone is preferably acetone. The precipitate may be combined with an adsorption salt to stabilize the final product. Suitable adsorption salts include, for example, calcium phosphate (CaHPO₄.2H₂O) and/or dihydrate calcium sulphate (CaSO₄.2H₂O). The resulting isolated polysaccharide may be formulated by mixing with an animal feed carrier in a dosage formulation that is effective to reduce growth of non-beneficial microorganisms in the digestive tract of a predetermined animal.

In one embodiment, starting materials include commercial pasteurized and spray-dried standard food grade Candida utilis that is subjected to the preferred process described below:
1.1 Weigh approximately 100 g of soy bean seeds. Soak them for 24 hrs in water.
1.2 Wash the seeds several times with water.
1.3 Grind the seeds in a mortar or a mincer.
1.4 Prepare an aqueous solution of 2 l containing 6.25 g/l of MnSO₄.H₂O at a temperature of 37° C. Add, stirring in a magnetic stirrer, 0.21 g/l of MnO₂, 0.6 g/l camphor, 62.5 g/l of desiccated C. utilis and 12.5 g/l of the seed milling.
1.5 Incubate in orbital stirrer at 37° C. and 200 rpm 2 to 5 hours, until the concentration of the polysaccharide is between 3 to 4 g/l.
1.6 Cool to a temperature less than 25° C., allow to stand, separate the supernatant and filter through a Hyflo®/Standar super cell® with a filter candle.
1.7 Concentrate the filtrated supernatant by ultrafiltration with a cut off of 20 kDa to a 1/10 of the original volume.
1.8 Diafiltrate the concentrate against at least 10 times of its volume of water.
1.9 Add, under stirring, calcium chloride to the concentrate/diafiltrate to a end concentration of 60 mM. Let, under stirring, 30 minutes.
1.10 Add, under stirring, calcium phosphate to a end concentration similar to three times the polysaccharide concentration. Let, under stirring, 15 minutes.
1.11 Add, under stirring, acetone to an end concentration of 40% (v/v).
1.12 Filter through nylon and separate the precipitate.
1.13 Dry the precipitate in a vacuum oven at temperature not higher than 55° C.

The above process is scalable to industrial level and implies an improvement respect to the prior art in the following points:
a) cobalt chloride is advantageously not needed.
b) filtration replaces centrifugation where filtration is a less expensive and more scalable process.
c) the former lyophilization may be replaced by adsorption on a salt, such as calcium phosphate, with precipitation. This renders a more stable product, due to the stabilizing action of the calcium phosphate.

### EXAMPLE 2

### Feeder Animal Study

Swine feed studies may be performed on a contract basis, for example, between a requesting agency and a testing agency. In one example, a study may be commissioned using two different test articles, namely: (1) glucomannan and (2) glucomannan plus a non-covalently linked protein. The test articles may be mixed in swine starter and grower feeds at varying concentrations for example: 1 mg/kg, 3 mg/kg and 20 mg/kg. A study of this type would show that swine fed either type of test article would perform better than the negative control having no antibiotic in feed, and as well as or better than positive control with antibiotic in the feed. Possible parameters used for comparison of the test articles to negative and positive controls may include, for example, total weight gain, weekly weight gain, feed conversion, mortality, carcass weight, bacterial flora, blood chemistry, and peripheral blood cell populations.

### Experimental Design

Study Summary

The two prebiotics including Candida utilis phosphoglucomannan and Candida utilis phosphoglucomannan-soybean proteins would be mixed prior to study initiation with a carrier, such as (CaHPO₄.2H₂O) and/or dihydrate calcium sulphate (CaSO₄.2H₂O). The negative control would be considered to have 0 mg test article/kg diet. As an example, the test articles would be titrated into the negative control feed at levels to approximate 1, 3, and 20 mg of active test article/kg body weight. BMD 60¹ would be added to the negative control diet at one pound per ton diet and this would be considered the positive control ration. Thus, there would be 8 treatment groups with 26 swine/group. Each treatment group would be divided into 2 pens of 13 swine, designated Replicate A and Replicate B. The eight rations would be fed ad libitum to 2 pens of 13 swine each for the duration of the study. Body weight, feed consumption and feed efficiency would be measured weekly and feed efficiency corrected for any mortality.
¹BMD 60 contains Bacitracin at 60 mg/lbs. BMD 60 produced by Carl S. Akey, Inc. PO Box 5002, Lewisburg, Ohio 45338.

The study described below may be replicated in relevant time intervals for swine in any one of nursery, feeder or maintenance stages, or a combination of these stages. The study below is commissioned for the feeder stage.

Blood will be collected weekly from 3 predetermined swine from Replicate A of each treatment group and submitted for CBC/Chemistries. Additionally, six (6) swine per treatment group (3 per pen) will be sacrificed on Day 90 for CBC & Chemistries. At the conclusion of the study, gut samples will be taken from three of swine from each treatment group (Replicate B) and sent off to determine levels of Salmonella spp. and Campylobacter spp. present.

Justification for Route, Duration

It is common practice in the swine industry to include antibiotics and other growth promoters in the feed of the swine; this is the most cost efficient method. The duration of the study is designed to mimic a standard growth phase commonly found in the feeder swine industry.

### Justification for Test Animal Selection

These products are designed to replace antibiotics at sub therapeutic levels in feeder swine.

Justification for Number of Animals

The number of animals in the protocol is considered to be the minimum necessary to evaluate the effects of the test articles in comparison to sub therapeutic doses of antibiotics in feeder swine.

Justification for Dose Selection

The current dose levels for the two test articles will be 0, ˜1 mg active test article/kg body weight, ˜3 mg active test article/kg body weight, and ˜20 mg active test article/kg body weight. These doses are considered to be safe doses for the two prebiotics including the aforementioned Candida utilis phosphoglucomannan and Candida utilis phosphoglucomannan-soy bean proteins.

### Study Outline

Effective Area

Sub Therapeutic Antibiotic Replacement

### Test Articles

Test Article 1 (Candida utilis phosphoglucomannan adsorbed in calcium phosphate). Candida utilis phosphoglucomannan 10-13% (w/w), dihydrated calcium phosphate 87-90% (w/w). This compound may be prepared, for example, by Industrial Farmaceutica Cantabria and provided to a test agency prior to study initiation.

Test Article 2 (Candida utilis phosphoglucomannan-soy bean proteins adsorbed in calcium phosphate—calcium sulphate). Candida utilis phosphoglucomannan—Soy bean proteins 5-10% (w/w), dihydrated calcium phosphate dihydrated calcium sulphate 90-95% (w/w). This compound may be prepared by Industrial Farmaceutica Cantabria and provided to the test location prior to study initiation.

Identification of Test Articles

Unless otherwise noted, the identity, strength, purity, composition, stability and method of synthesis, fabrication and/or derivation of each batch of the test and control articles is documented by the test agency before its use in the study. This documentation is maintained by the test agency.

Archival Samples

An archival sample from each lot of test article is taken and stored in the Archives of the test agency, pending shipment to the requesting agency.

Preparation of Test Diets

Given the desired dose (approximate) levels of the test articles of 1, 3, and 20 mg active test article/kg body weight, the average ratio of grams feed intake/day/Kg body weight is taken from the NRC (1994) for the swine of age 1 to 3 weeks and 3 to 9 weeks. This value is used to determine the mg total product/Kg feed to mix for each treatment group and time period. The following Table 6 outlines the values that may be used for each treatment:

TABLE 6
Dosing Study

| | | | | Dose Levels (Active | | | |
|---|---|---|---|---|---|---|---|
| | | Pos Cont. | | Article per Test Article) | | | |
| | | addition | | | ˜3 mg | | |
| Test | | mg/kg | Neg | ˜1 mg per | per Kg | ˜20 mg per | |
| Article | | feed | Cont. | Kg Feed | Feed | Kg Feed | Comments |
| Candida | N = 26* | 499.4 | 0.0 | 88.63 | 265.9 | 1772.64 | 0-3 Week |
| utilis-Phos | | | | | | | |
| (10% | | | | | | | |
| active) | | | | | | | |
| Candida | N = 26* | — | — | 125.24 | 375.71 | 2504.72 | 3-7 Week |
| utilis-Phos | | | | | | | |
| (10% | | | | | | | |
| active) | | | | | | | |
| Candida | N = 26* | — | — | 177.26 | 531.79 | 3545.28 | 0-3 Week |
| utililis- | | | | | | | |
| Phos + | | | | | | | |
| Soy (5% | | | | | | | |
| active) | | | | | | | |
| Candida | N = 26* | — | — | 250.47 | 751.42 | 5009.44 | 3-7 Week |
| utililis- | | | | | | | |
| Phos + | | | | | | | |
| Soy (5% | | | | | | | |
| active) | | | | | | | |

*Six swine per Tx group are sacrificed on day 3, N = 20 per Tx group thereafter

Table 6 shows the dosing levels for each test article. Table 6 lists the amount of test article to add based on the required dose level of the active article consumed per kilogram of feed consumed. The swine are fed suitable amounts of food for their age and size to meet the study dosing requirements.

Analysis of Test Diets

Due to the nature of the test articles there is currently no accurate methodology to quantitate the amount of test article or its activity in the test diets other than an empirical study, for example, as described herein.

Preparation of Facilities

Prior to the receipt of the swine the facility is cleaned and sanitized removing all organic matter. Each pen is set up so as to isolate it from all other pens; this is done in order to prevent possible cross contamination among pens. Each pen is uniformly provided with suitable equipment for the raising of swine.

Acquisition of Animals

250 conventional feeder swine are obtained from a commercial breeder.

### General Husbandry

Housing:

Swine are housed in an environmentally controlled room at the test agency for the duration of the study.

Feeding During Acclimation Phase:

During the acclimation phase (days −6 to −1) all swine are fed ad libitum the negative control diet, containing no test article or antibiotic.

Feeding During Trial Phase:

Each pen is initially fed a suitable amount of the designated ration. The feed intake is observed daily and feed is weighed and added as necessary in order to insure the swine are maintained on ad libitum feeding.

Animal Identification

At approximately day −6 of the experimental phase all swine are ear tagged with a unique number identification in the right ear.

Animal Selection at day 0 of Experimental Phase

At day 0 of the experimental phase all swine are individually weighed. Swine selection and randomization procedures is conducted by test agency personnel (other than the Investigator or Co-Investigator) using Microsoft® Excel 2002 (10.4524.4219) SP-2. Random numbers are generated using the “Rand” function of Excel and are captured using the “copy/paste special/values” commands. The “Rank” function in Excel is used to assign swine to groups within blocks by random number. In addition, single factor ANOVA data analysis (α=0.05) in Excel is used to assess the outcomes of randomizations for homogeneity of variance (F statistic<F critical value) between groups. ANOVA is conducted for body weight between pens.

Swine Selection for Blood Draw

Each pen has an additional 3 swine (N total swine=13 per pen) included at Day 0 to provide 3 swine per pen (6 per treatment) on Day 3 for sacrifice and blood collection. Selection of the 3 swine for the Day 3 sacrifice is by a random number assignment. All thirteen swine in each pen receive a random number generated in Excel. The three swine with the highest random numbers within each pen are selected for the Day 3 collection.

Ten (10) swine remain in each pen to complete the study. Three (3) swine from the Replicate A pen of each treatment group are selected for blood draw each week of the experimental phase. Within Replicate A, swine are selected for blood collection based on their random numbers. The swine with the lowest 3 random numbers are drawn on weeks 1, 4, and 7, the next 3 lowest on weeks 2, and 5, and the next 3 lowest on weeks 3 and 6. The tenth swine within the replicate is considered an extra swine.

Unused Test Articles

Unused test article mixtures and containers are returned to the requesting agency. Collection equipment used in the study are autoclave and disposed of in the biohazard/sharps solid waste stream at the test agency.

### Test Animals

Species

Feeder Swine

Supplier

Any commercial breeder capable of supplying a group of animals having a uniform breeding standard.

Animal Requirements/Specifications

Number of Animals on Study

| Total* | Males* | Females* |
|---|---|---|
| 208 | 208 | 0 |

Age

30-120 days of age

Acclimation Period

At this stage, the feeder swine begin acclimation to study conditions at about 5 to 7 days prior to the initiation of the trial. During acclimation, all swine are checked for viability twice daily. Prior to assignment to study, all swine are examined to ascertain suitability for study by a staff veterinarian.

Clinical Observations

At approximately day −7 of the study clinical observations are made by a staff veterinarian for each swine. Any swine that is found abnormal is rejected from the study.

3.4.2 Identification

At approximately day −6 of the study all swine are individually ear tagged with a unique numerical identification in the right ear. At approximately day 30 the swine are given an additional ear tag in the left ear, this is the same number as was placed in the right ear at day approximately −6.

### Animal Care and Husbandry

Facilities Management/Animal Husbandry

Currently acceptable practices of good animal husbandry are followed, e.g., as shown in the Guide for the Care and Use of Laboratory Animals; National Academy Press, 1996. The test agency, for example, may be fully accredited to perform contract studies by the Association for Assessment and Accreditation of Laboratory Animal Care International (AAALAC). Suitable standards are imposed, for example, to provide space requirements for the growth of swine at the feeder stage.

Veterinary Care

Swine are monitored by the technical staff for any conditions requiring possible veterinary care. If any such conditions are identified, a staff veterinarian is notified for an examination and evaluation.

### Environmental Conditions

Light/Dark Cycle

During the study the swine are uniformly provided with ambient lighting that is know to be suitable for the production of swine.

Temperature

During the study the swine are uniformly provided with ambient temperature that is known to be suitable for the production of swine.

Humidity

Humidity is monitored in accordance with standard procedure at the test agency, but is not controlled.

Housing

The swine are housed in groups of 10 in individual floor pens in an environmentally controlled room for the duration of the study.

Feed

Swine are allowed ad libitum feeding. From days −6 to 0 all swine are given the negative control diet (containing no test articles or antibiotics). From day 0 forward each pen is given its respective diet ad libitum.

Water

Clean, fresh water from an on-site deep well is available ad libitum during the study.

Bedding

An approximate mixture of 50/50 (v/v) of fresh straw and straw that has been previously used for feeder swine bedding is used in this trial. The purpose of the litter contamination is to increase the pathogen burden in the test swine to better reflect the normal farm husbandry condition. It is also desirable to have a pressure of infection to determine the efficacy of the test article.

Feed Analysis

Nutritional certification of batches of feed provided by the manufacturer (via manufacturer's bag label) is included in the raw data. There are no known contaminants in the food which are expected to interfere with the objectives of this study.

Water Analysis

A copy the test agency's most recent water analysis is included with the raw data. There are no known contaminants which are expected to interfere with the objectives of this study.

### In-Life Evaluations

### Observations

Body Weight Gain

Each swine is weighed once a weekly, this information is recorded in the study records.

Feed Intake

Feed is weighed out prior to feeding. All feed added to a pen is weighed and recorded in the study records. Once weekly the feeders is weighed and weights recorded in order to determine feed disappearance.

Environmental

Once daily the minimum, maximum and current temperature and humidity are recorded in the study records.

Mortality

Mortality is recorded daily for each pen in the study book. The body weight is recorded for each mortality and recorded in the study book.

Euthanasia

The feeder swine are euthanized by an intravenous overdose of sodium pentobarbital (390 mg/mL)/sodium phenylion (50 mg/mL) at 0.22 mL/Kg, followed by cervical dislocation (e.g., as SRC SOP PR.04.01).

Blood Collection.

Blood is collected for determination of CBC with differential and Chemistries on Study Days 3, 7, and weekly thereafter. The samples are collected by test agency personnel and sent to a suitable analytical company, such as Antech Diagnostics for analysis. For the day 3 draw the swine are sacrificed and blood is collected via a direct heart draw. From Days 7 on the blood is collected from the brachial artery. For the CBC approximately 1 mL of whole blood is drawn using a drop for the blood smear and the rest drawn into an EDTA microtainer for storage and reuse. The differential for the CBC is automated. The analytical chemistry requires approximately 0.50 mL serum from each swine.

Bacteriology

On day 90 of the study 3 swine from each pen are sacrificed for gut collection. A sample of the small intestine is collected from each swine, from the ileum-cecal junction to the Meckel's Diverticulum. Sub samples from this portion of the small intestine is taken and sent to Antech Diagnostics Laboratories to determine Salmonella spp. and Campylobacter spp. counts. This data is recorded in the study records.

Carcass Meat Yield

On day 90 of the study all swine are sacrificed and the carcass is weighed. This data is recorded in the study records

Gross Necropsy/Gross Pathology

On day 90 of the study all swine are sacrificed and a staff Veterinarian performs a gross necropsy and gross pathology. These results are included in the study report.

Archiving of Records and Specimens

All data documenting experimental details and study procedures and observations are recorded and maintained as raw data. At the completion of the study, all reports and study specific original raw data, and copies of certain study related facility data are reported. An exact copy of the report and raw data is maintained in the test agency's archives for a period of at least 1 year after submission of the signed final report. All plasma samples are shipped to the test requestor. The test requestor is responsible for retaining samples of the test article.

### Statistical Analysis

ANOVA statistical analysis is performed on study data including Body Weight Gain, Feed Consumption, Feed Efficiency corrected for mortality, and carcass meat yield. Alpha is set at 0.05.

Those skilled in the art will appreciate that the foregoing description teaches by way of example, and not by limitation. Accordingly, what is shown and described should be construed in a manner that is consistent with the scope and spirit of the invention.

### REFERENCES

The following documents are incorporated by reference to the same extent as though fully replicated herein:
¹ Choct M C (2001) Alternatives To In-Feed Antibiotics In Monogastric Animal Industry. ASA Technical Bulletin Vol. AN30-2001 p. 1-6
² Mathew A (2002) Seeking Alternatives to Growth Promoting Antibiotics. Depart. Of Animal Science, The Uni. Of Tennessee, Knoxville Tenn., USA
³ Turner J L, Pas, Dritz S S, and Minton J E (?) review: Alternatives to Conventional Antimicrobials in Swine Diets. The Professional Animal Scientist 17. p. 217-226
⁴ Mitchener B (1999) EU Moves Toward a Total Ban of Antibiotics in Animal Feed. Wall Street Journal, Jul. 28, 1999
⁵ Newman K (1994). Mannan-oligosaccharides: Natural Polymers with significant impact on the gastrointestinal microflora and the immune system. In: Lyons T P and J, K A (ed.) Biotechnology in the Feed Industry. Nottingham University Press, Nicholasville, Ky., p. 167-180
⁶ Waldroup P W, Oviedo-Rondon E O, fritss CA (2003) Comparison of Bio-Mos and Antibiotic Feeding Programs in Broiler Diets Containing Copper Sulfate. International Journal of Poultry Science 2 p 28-31
⁷ Fritts C A and Walroup P W (2003) Evaluation of Bio-Mos® Mannan Oligosaccharide as a replacement for Growth Promoting Antibiotics in Diets for Turkeys. International Journal of Poultry Science 2 p 19-22
⁸ Parks C W, Grimes J L, Ferket P R, and Fairchild A S (2001). The Effect of Mannanoligosaccharides, Bambermycins, and Virginiamycin on Performance of Large White Male Market Turkeys. Poultry Science 80 p 718-723
⁹ LeMieux F M, Southern L L, and Bidner T D (2003) Effect of mannan oligosaccharides on growth performance of weanling pigs. J. Animal Sci. 81 p 2482-2487
¹⁰ Davis M E, Maxwell C V, Brown D C, de Rodas B Z, Johnson Z B, Kegley E B, Hellwig D H, and Dvorak R A (2002) Effect of dietary mannan oligosaccharides and(or) pharmacological additions of copper sulfate on growth performance and immunocompetence of weanling and growing/finishing pigs. J. Animal Sci. 80 (2887-2894)
¹¹ Davies M E, Maxwell C V, Erf G F, Brown D C, and Wistuba T J (2004). Dietary supplementation with phosphorylated mannans improves growth response and modulates immune function of weanling pigs. J. Animal Sci. 82 p 1882-1891
¹² Franklin S T, Newman M C, Newman K E, and Meek K I (2005) Immune Paramteres of Dry Cows Fed Mannan Oligosaccharide and Subsequent Transfer of Immunity to Calves. J. Dairy Sci. 88 p 766-775
¹³ Buddington K K, Donahoo J B, Buddington R K (2002) Dietary Oligofructose and Inulin Protect Mice from Enteric and Systemic Pathogens and Tunor Inducers. P 472-477
¹⁴ Swanson K S, Grieshop C M, Flickinger E A, Bauer L L, Healy H P, Dawson K A, Merchen N R, and Fahey G C (2002) Supplemental Fructooligosaccharides and Mannanoligosaccharides Influence Immune Function, Ileal and Total Tract Nutrient Digestibilities, Microbal Populations and Concentrations of Protein Catabolites in the Large Bowel of Dogs. Nutritional Immunology p 980-989
¹⁵ Fernandez F, Hinton M, and Van Gils B (2002) Dietary mannan-oligosaccharides and their effect on chicken caecal microflora in relation to Salmonella Enteritidis colonization. Avian Pathology 31 p 49-58.
¹⁶ Allen V M, Fernandez F, and Hinton M H (1997). Evaluation of the influence of supplementing the diet with mannose or palm kernel meal on salmonella colonization in poultry. British Poultry Science 38 p 485-488
¹⁷ Tizard R I, Carpenter R H, McAnalley B H, and Kemp M C (1989) The biological activities of mannans and related complex carbohydrates. Mol. Biother. 1 p 290-296
¹⁸ Krizkova L, Durackova Z, Sandula J, Sasinkova V, and Krajcovic J (2001) Antioxidative and antimutagenic activity of yeast cell wall mannans in vitro. Mutation Research 497 p. 213-222
¹⁹ Djeraba A and Quere P (2000) In vivo macrophage activation in chickens with Acemannan, a complex carbohydrate extracted from Aloe vera. International Journal of Immunopharmacology 22 p. 365-372
²⁰ Olivella J G, and Torrus E F (1997) Study of the immunostimulating effect of glycophosphopeptical (AM3) in mice. FEMS Immunology and Medical Microbiology 18 p. 87-89
²¹ Villarrubia V G, Moreno Koch M C, Calvo C, Gonzalez S, and Alvarez-Mon M. (1997) The immunosenescent phenotype in mice and humans can be defined by alterations in the natural immunity reversal by immunomodulation with oral AM3. Immunopharmacology and Immunotoxicology 19 p 53-74
²² Prieto A, Reyes E, Bernstein E D, Martinez B, et al (2001) Defective Natural Killer and Phagocyctic Activities in Chronic Obstructive Pulmonary Disease Are Restored by Glycophosphopeptical (Immunoferon). Am J Respir Crit Care Med Vol. 163 p 1578-1583
²³ Brieva A., Guerrero A, Pivel J P. (2002) Immunoferon, a glycoconjugate of natural origin, regulates the liver response to inflammation and inhibits TNF-α production by an HPA axis-dependent mechanism. Internation Immunopathology Vol. 275 p
²⁴ Brieva A, Guerrero A, Alonso-Lebrero J L, Pivel J P (2001). Immunoferon, a glycoconjugate of natural origin, inhibits LPS-induced TNF-α production and inflammatory responses. International Immunopharmacology Vol 1 p 1979-1987
²⁵ Johnson R W, (1997). Inhibition of Growth by Pro-Inflammatory Cytokines: An intergrated View. J Anim Sci Vol 75 p 1244-1255
²⁶ Podzorski R P, Gray G R, and Nelson R D (1990) Different Effects of Native Candida albicans Mannan and Mannan-Derived Oligosaccharides on Antigen-Stimulated Lymphoproliferation In Vitro. The Journal of Immunology Vol. 144 P 707-716.

1. A method of supplementing a swine diet, the method comprising the steps of:
mixing swine feed with a phosphorylated glucomannan polysaccharide in an effective amount to benefit swine production, in order to provide a mixed swine feed.

2. The method of claim 1, further comprising a step of feeding the mixed swine feed to swine to obtain a swine production benefit from use of the phosphorylated glucomannan polysaccharide.

3. The method of claim 2, wherein the swine production benefit includes at least one benefit selected from the group consisting of: increased swine weight gain, increased relative quantities of the beneficial bacteria in the swine, decreased relative quantities of malicious bacteria in the swine, increased uptake of beneficial minerals, nutrients and vitamins; increased uptake of zinc and copper, improved overall general health of the swine, and combinations thereof.

4. The method of claim 3, wherein the swine production benefit includes increased muscle mass.

5. The method of claim 1, wherein the phosphorylated glucomannan contains a repeating polysaccharide subunit that is repeated approximately n times of 1-6 and 1-2 linkages between and within mannose and glucose residues at a ratio of 12:1 mannose:glucose, were n ranges from 10 to 40.

6. The method of claim 5, wherein n ranges from 10 to 20.

7. The method of claim 5, wherein n ranges from 20 to 30.

8. The method of claim 5, wherein n ranges from 30 to 40.

9. The method of claim 5, wherein n ranges from 20 to 40.

10. The method of claim 5, wherein the phosphorylated glucomannan is complexed with a protein.

11. The method of claim 10, wherein the phosphorylated glucomannan and protein are combined with a matrix or carrier.

12. The method of claim 11, wherein the matrix or carrier is inorganic.

13. The method of claim 5, wherein the phosphorylated glucomannan is combined with a matrix or carrier.

14. The method of claim 11, wherein the matrix or carrier is inorganic.

15. The method of claim 1, wherein the swine production benefit is at least selected from the group consisting of reducing the subtherapeutic dose of antibiotic needed to accelerate weight gain; eliminating subtherapeutic doses of antibiotic in the starting and growing of feeder swine, and eliminating subtherapeutic doses of antibiotics in the starting and growing of swine.

16. The method of claim 1, wherein the step of mixing includes combining ingredients to form a liquid, gel, or colloid.

17. The method of claim 1, wherein the step of mixing includes combining ingredients to form a solid.

18. The method of claim 1 wherein the step of mixing includes combining ingredients that include a predetermined formulation of nutrients that target a specific stage of swine development.

19. In a swine feed, the improvement comprising:
a phosphorylated glucomannan polysaccharide mixed with the swine feed in an effective amount to benefit swine production.

20. The swine feed of claim 19, wherein the swine feed is formulated for optimal benefit at a nursery stage of swine development.

21. The swine feed of claim 19, wherein the swine feed is formulated for optimal benefit at a feeder stage of swine development.

22. The swine feed of claim 19, wherein the swine feed is formulated for optimal benefit of a maintenance stage of swine development.

23. The swine feed of claim 19, wherein the effective amount includes an amount ranging from 1 mg to 5 mg per kg of body weight based upon a targeted intake of food for the swine.
